(19) United States
(12) Patent Application Publication
(10) Pub. No.: US 2024/0350390 A1
(43) Pub. Date: Oct. 24, 2024

(54) HUMAN MILK OLIGOSACCHARIDES IN SULFATE-FREE CLEANSING COMPOSITIONS

(71) Applicant: DSM IP ASSETS B.V., Heerlen (NL)

(72) Inventors: Lise Anne KOHLER, Kaiseraugst (CH); Pascal Christian SIEBER, Kaiseraugst (CH)

(21) Appl. No.: 18/683,029

(22) PCT Filed: Aug. 23, 2022
(86) PCT No.: PCT/EP2022/073482

§ 371 Date: Feb. 12, 2024

(30) Foreign Application Priority Data
Aug. 24, 2021 (EP) 21192699.3

(51) Int. Cl. A61K 8/60; A61K 8/44; A61Q 19/00; A61Q 19/10

(52) U.S. Cl. CPC A61K 8/608; A61K 8/442; A61Q 19/005; A61Q 19/10; A61K 2800/30

(57) ABSTRACT

The present invention relates to sulfate-free cleansing compositions comprising at least one zwitterionic surfactant and at least one human milk oligosaccharide as well as to a method of increasing the foaming properties of cleansing compositions comprising at least one surfactant by incorporation of one or more human milk oligosaccharides into said cleansing composition.

The present invention relates to sulfate-free cleansing compositions comprising at least one zwitterionic surfactant and at least one human milk oligosaccharide as well as to a method of increasing the foaming properties of cleansing compositions comprising at least one surfactant by incorporation of one or more human milk oligosaccharides into said cleansing composition.

Sulfate-based surfactants, i.e. surfactants including one or more sulfate groups such as for example sodium lauryl sulfate or sodium laureth sulfate are commonly used, highly effective surfactants found in many mainstream cleansing products including shampoos, soaps, detergents, toothpastes and body washes. In addition, these compounds are known to provide good foaming properties. Said sulfate-based surfactants are however also known to be rather harsh on the skin and to cause varying levels of skin and eye irritation. Furthermore, said sulfates-based surfactants often dry out the skin, and some people find they may lead to more acne when their skin is in frequent contact therewith. Thus, consumer demand is more and more shifting to cleansing products that are free of such sulfate-based surfactants, in particular for sensitive skin.

In a number of cleansing applications, consumers are looking for a high foaming capacity. For example, a shampoo that does not produce enough creamy, stable foam during shampooing has no chance of success on the market. The same applies to manual dishwashing detergents, even though a direct connection between the foaming capacity and the cleaning performance cannot be established in many cases.

Hence, besides performance requirements, such as cleaning performance and dermatological compatibility, the foaming behavior is a further important product feature of cleansing products.

Thus, there is an ongoing need for cleansing compositions, which do not comprise sulfate-based surfactants but nevertheless exhibit excellent foaming properties.

Surprisingly it has now been found that human milk oligosaccharides (HMOs) are able to significantly increase the foaming characteristics of sulfate-free cleansing compositions.

Thus, in a first aspect, the present invention relates to sulfate-free cleansing compositions comprising at least one surfactant, preferably one zwitterionic surfactant comprising at least 8 carbon atoms and one or more human milk oligosaccharide.

In a second aspect the present invention relates to a method of increasing the foaming properties of sulfate-free cleansing compositions comprising at least one surfactant, said method comprising the step of adding at least one human milk oligosaccharide into said cleansing composition and optionally appreciating the effect. Preferably, said at least one surfactant comprises a zwitterionic surfactant comprising at least 8 carbon atoms.

In a third aspect, the present invention relates to the use of at least one human milk oligosaccharide as foam-enhancing agent or foam booster, in particular in sulfate-free cleansing compositions comprising at least one surfactant. Preferably, said at least one surfactant comprises a zwitterionic surfactant comprising at least 8 carbon atoms.

In a further embodiment, the present invention relates to a method for improving the foaming properties of sulfate-free surfactants in cleansing compositions, which method comprises combining at least one human milk oligosaccharide with said surfactant(s).

Preferably, said sulfate free surfactants encompasses at least one zwitterionic surfactant comprising at least 8 carbon atoms.

The term ‘cleansing composition’ as used herein refers to compositions suitable for cleansing. Such compositions are characterized by comprising at least one surfactant and encompass shampoos, body washes, toothpastes and the like.

The term ‘sulfate-free’ as used herein refers to cleansing compositions which do not comprise sulfates, i.e. do not comprise any organic compounds that include a sulfate moiety (—OSO₃²⁻) such as e.g. laureth sulfates or lauryl sulfates; a composition may however include sulfonates, and still be considered “sulfate-free” or “free of sulfates” as those terms are used herein. It is well understood that in particular no sulfate-based surfactants i.e. surfactants comprising sulfate groups such as sodium or ammonium lauryl sulfate or sodium or ammonium laureth sulfate are present in the cleansing compositions according to the present invention.

The term ‘foam-enhancing agent’ respectively ‘foam booster’ as used herein refers to an agent which favorably influences the foam properties of a surfactant and/or a cleansing composition with regard to foaming ability, foam stability (foam remaining after certain period at rest), foam quantity (volume/height) (associated with good cleaning effect), creaminess of the foam (associated with conditioning effect), foam density, texture of the foam and/or foam speed (foam produced after a very short period of time. In particular the term ‘foam-enhancing agent’ respectively ‘foam booster’ is to be understood to enhance the foam quantity (volume/height) compared to a control, i.e. in the absence of human milk oligosaccharide(s). In particular, the foam quantity (volume/height) increase according to the present invention is greater than 5%, preferably greater than 10%, more preferably greater than 30% as measured according to the cylinder shake method compared to control, i.e. the respective composition not comprising the HMO(s).

The cylinder shake method is performed by preparing an aqueous composition comprising at least one surfactant with/without one or more human milk oligosaccharide(s) (also referred to as HMO(s)), placing the test composition in a cylinder and stoppering the cylinder, agitating the test composition by vigorously shaking the tube vertically for a set period of time (15 s), and immediately measure the height (volume) of the foam in the cylinder at the completion of the agitation step.

Increased foam height (volume) compared to control, i.e. the test sample without HMO(s) indicates increased foam power.

The term ‘fatty’, as used herein, preferably refers to a hydrocarbon chain having 12-22 carbon atoms (C₁₂₋₂₂). The chain may be straight or branched and may be saturated or unsaturated (typically one or two double bonds in the chain).

The term ‘human milk oligosaccharides’ (HMOs) refers to a family of structurally diverse unconjugated glycans that are highly abundant in and unique to human milk.

Originally, HMOs were proposed to be prebiotic “bifidus factors,” or human milk glycans found to promote growth in Bifidobacterial species of the gut and found uniquely in the stool of breast fed infants compared to formula fed infants. HMOs are composed of the five monosaccharides glucose (Glc), galactose (Gal), N-acetylglucosamine (GIcNAc), fucose (Fuc) and sialic acid (Sia), with N-acetylneuraminic acid (Neu5Ac) as the predominant if not only form of Sia. More than two hundred different HMOs have been identified so far. The most important ones are 2′-fucosyllactose (2′FL), lacto-N-neotetraose (LNnT), 3-fucosyllactose (3FL), difucosyllactose (DFL), Lacto-N-fucopentaose I (LNFP I), 3′Sialyllactose Sodium Salt (3′SL), 6′Sialyllactose Sodium Salt (6′SL), and Lacto-N-Tetraose (LNT).

HMOs can be isolated from breast milk or they can be produced chemically or biochemically. HMOs are available commercially from a variety of producers.

For the purpose of the present invention the source of the HMO is not essential. It is clear that HMOs from different sources can be used.

Particularly suitable HMO's in all embodiments of the present invention are fucosylated HMO's such as in partivuar α1-2 respectively α1-3 fucosylated HMO's; sialylated HMO's such as in particular sialyllactoses, as well as lacto-N-(neo)tetraoses as well as any mixtures thereof.

Particularly preferred fucosylated HMO's according to the present invention are 2′-fucosyllactose (CAS No: 41263-94-9), 3-fucosyllactose (CAS No: 41312-47-4), difucosyllactose (also known as Lactodifucotetraose; CAS No: 20768-11-0) and Lacto-N-fucopentaose I (CAS No: 7578-25-8).

Particularly preferred sialylated HMO's according to the present invention are sialyllactoses as well as salts thereof (preferably the sodium salts) such as in particular 3′sialyllactose and 6′sialyllactose as well as the respective sodium salts thereof (CAS No's: 35890-39-2 (3′sialyllactose); 128596-80-5 (3′sialyllactose sodium salt); 35890-39-2 (6′sialyllactose); 157574-76-0 (6′sialyllactose sodium salt)).

Particularly preferred lacto-N-(neo)tetraoses according to the present invention are lacto-N-tetraose (CAS No: 14116-68-8) and lacto-N-neotetraose (CAS No: 13007-32-4), which are highly abundant neutral core HMO's in human milk.

The total amount of the at least one HMO used according to the present invention is preferably at least 0.1 wt.-%, more preferably at least 0.25 wt.-%, most preferably at least 0.5 wt.-% and/or up to 10 wt.-%, more preferably up to 5 wt.-%, most preferably up to 2.5 wt.-%. Even more preferably, the amount is selected in the range from 0.01 to 10 wt.-%, more preferably in the range from 0.1 to 7.5 wt.-%, most preferably in the range from 0.2 to 5 wt.-%, based on the total weight of the cleansing composition. Further suitable ranges are from 0.25 to 2.5 wt.-% and from 0.5 to 2 wt.-%. Particularly preferred ranges according to the present invention are from 0.1 to 1 wt.-%, 0.2 to 1 wt.-% as well as 0.5 to 1 wt.-%, such as from 0.25 to 0.75 wt.-% or from 0.3 to 0.5 wt-%.

In the embodiment of the present invention the HMO may not only serve as a foaming agent but may also excerpt positive effects on the skin.

Preferably in all embodiments of the present invention the at least one HMO is selected from the group consisting of 2′-fucosyllactose (2′ FL), 3-fucosyllactose (3FL), difucosyllactose (DFL), lacto-N-fucopentaose I (LNFP-1), 3′sialyllactose sodium salt (3′SL), 6′sialyllactose sodium salt (6′SL), lacto-N-neotetraose (LNnT), Lacto-N-tetraose (LNT), as well as mixtures thereof, more preferably from 3′Sialyllactose Sodium Salt (3′SL), 3-fucosyllactose (3FL), 6′sialyllactose sodium salt (6′SL), lacto-N-neotetraose (LNnT), Lacto-N-tetraose (LNT), difucosyllactose (DFL) as well as mixtures thereof, most preferably from 3′sialyllactose sodium salt (3′SL), 3-fucosyllactose (3FL) and lacto-N-neotetraose (LNnT). In all embodiments the use of LNnT, LNT and the mixture of 2′-FL/DFL is particularly preferred.

The sulfate-free surfactant according to the present invention can be non-ionic, anionic, cationic or zwitterionic. It is well understood that the cleansing composition may include more than one different type of surfactant, which may each independently be selected from sulfate-free non-ionic, anionic, cationic or zwitterionic surfactants.

Preferably, the compositions according to the present invention comprise at least one zwitterionic surfactant comprising at least 8 carbon atoms.

Particularly suitable non-ionic surfactants to be used for the purpose of the present invention encompass alkyl (poly)glucosides, i.e. compounds of the general formula CₙH₂ₙ₊₁O(C₆H₁₀O₅)ₓH, with x 1 to 4 such as preferably decyl glucoside and lauryl glucoside; polyethyleneglycol (PEG) based surfactants such as in particular mono- and di-esters of fatty acids with polyethylene glycol such as PEG-8 laurate, PEG-8 dilaurate, PEG-100 stearate and PEG-150 distearate as well as ethoxylated fatty acid glycerides (PEG-n glyceryl acylates) such as PEG-4 castor oil, PEG-120 glyceryl stearate, triolein PEG-6 esters, PEG-200 hydrogenated glyceryl palmate and PEG-7 glyceryl cocoate; (poly)glyceryl fatty acid esters such as glyceryl myristate, glyceryl stearate, glyceryl palmitate lactate; ethoxylated glycerides; glycol esters and derivatives, mono-esters of either ethylene or propylene glycol such as preferably glycol acylate or propylene glycol acylate; ethers comprising aliphatic (C₆-C₁₈) primary or secondary linear or branched chain acids, alcohols or phenols which possess no functional grouping other than the terminal OH group of the Polyoxyethylenated (POE) chain as well as ethoxylated alcohols and propoxylated POE ethers such as preferably PEG ethers, PPG ethers, propylene glycol alkyl POE-n ethers; alkanolamides such as preferably N-acyl derivatives of monoethanolamine (MEA) and diethanolamine (DEA), ethoxylated or not; such as preferably PEG-n acylamides, coco mono- or di-ethanolamide, palmamide MEA, acylamide DEA; sorbitan/sorbitol esters such as acetylated sorbitan ethoxylated or not, polysorbate-n, sorbitan sequiisostearate; alkyl carbohydrates esters or sucrose esters resulting from trans-esterification of sucrose with fatty acid methyl esters or triglycerides such as preferably alkylpolysaccharides; amine oxides such as preferably cocoamidopropyl amine oxide and lauramine oxide.

The term zwitterionic surfactant (i.e. surfactants having at the same time a positive and a negative charge, also referred to as amphoteric surfactants) as used herein refers to surfactants, which are characterized by having at least 8 carbon atoms. Particularly suitable zwitterionic surfactants according to the present invention encompass secondary or tertiary aliphatic amine derivatives with an aliphatic chain, linear or branched, containing at least 8 to 22 carbon atoms and one anionic group selected from the group of carboxylate, sulfonatephosphate or phosphonate; acyl/dialkyl ethylenediamines such as preferably acylamphoacetate, disodium acylamphodipropionate, sodium acylamphohydroxypropylsulfonate, disodium acylamphodiacetate, sodium acylamphopropionate and wherein the acyl group represents either an alkyl oralkenyl group which can be mono- or polyunsaturated and contains from 5 to 29 carbon atoms; N-alkyl amino acids or imino diacids such as preferably aminopropyl alkylglutamide, alkylaminopropionic acid, sodium alkylimino propionate, alkyl glycinates and carboxyglycinates, sodium cocoglycinates; betaines such as preferably alkyl (C₈-C₂) betaines, alkyl amidopropyl betaines (cocamidopropyl betaines), alkyl (C₈-C₂₀)amidoalkyl (C₁-C₆) betaines, alkyl sulphobetaines and alkyl (C₈-C₂₀)amidoalkyl (C₁-C₆) sulphobetaines.

Particularly suitable anionic surfactants to be used for the purpose of the present invention are acyl isethionate salts such as preferably sodium acylisethionate, sodium cocoyl isethionate; alkylaryl sulfonates salts such as preferably sodium alkylbenzene sulfonate and/or sodium dodecylbenzene sulfonate; alkyl sulfonates salts such as preferably sodium alkenyl sulfonate (sodium C₁₂₋₄₀ olefin sulfonate), sodium alkylglyceride sulfonate (sodium cocomonoglyceride olefin sulfonate), sodium alkylether sulfonate (sodium C12-15 pareth-15 sulfonate) and/or sodium lauryl sulfoacetate; (di)sodium sulfosuccinates such as preferably sodium dialkyl sulfosuccinate (dioctyl sodium sulfosuccinate), disodium alkyl PEG-n sulfosuccinate, disodium alkylamido PEG-n sulfosuccinate (disodium oleamido MEA-sulfosuccinate), disodium alkylsulfosuccinate; alkyl phosphates (mono-esters) such as preferably TEA monolauryl phosphate; PEG-n alkyl phosphates such as preferably DEA oleth-10 phosphate; di PEG-n alkyl phosphates (di-esters) such as preferably dilaureth-4 phosphate; phospholipids (tri-esters) such as preferably lecithin; carboxylic acids ester, such as preferably mono-ester of di- or tri-carboxylic acids such as lactylates (sodium acyllactylate, calcium stearoyl lactylate), laureth-6 citrate, dinonoxynol-9 citrate; ether carboxylic acids such as preferably sodium PEG-n alkyl carboxylates, sodium trideceth-13 carboxylate, nonoynol-8 carboxylic acid, alkyl C₆-C₂₄ ether carboxylates polyoxyalkylenated; acyl glutamates such as preferably di-TEA palmitoyl aspartate and sodium hydrogenated tallow glutamate; Acyl peptides with various amino acids side groups such as preferably palmitoyl hydrolysed milk protein, sodium cocoyl hydrolysed soy protein, TEA-cocoyl hydrolysed collagen or other acyl hydrolysed protein salts; sarcosinates or acyl sarcosides such as preferably myristoyl sarcosine, TEA-lauroyl sarcosinate; as well as taurates and sodium methyl acyltaurates such as preferably sodium lauroyl taurate, sodium methyl cocoyl taurate as well as soaps.

Particularly suitable cationic surfactants according to the present invention encompass alkylamines such as preferably dimethyl alkylamine (dimethyl lauramine), dihydroxyethyl alkylamine dioleate, acylamidopropyldimethylamine lactate (cocamidopropyl dimethylamine lactate); alkyl imidazolines such as preferably alkyl hydroxyethyl imidazoline, Ethylhydroxymethyl oleyl oxazoline, alkyl aminoethyl imidazoline; ethoxylated alkylamines such as preferably PEG-n alkylamines, PEG-n Alkylaminopropylamine, poloxamine; quaternary compounds such as preferably tetraalkylammonium salts; alkyl trimonium chloride, PEG-n alkylmonium chloride, dialkyldimonium chloride (hydroxyethyl cetyldimonium chloride), alkylamidopropyl alkyldimonium tosylate (Cocamidopropyl ethyldimonium ethosulfate), PEG-n Acylmethyldiethonium methosulfate, dialkyl hydroxypropylmonium methosulfate, and alkyldimonium hydroxypropyl protein hydrolysate (Cocodimonium hydroxypropyl hydrolysed hair keratin).

In a preferred embodiment, the cleansing compositions according to the present invention comprise at least one non-ionic and at least one zwitterionic surfactant. Most preferably the cleansing compositions according to the present invention comprise solely non-ionic and zwitterionic (amphoteric) surfactants, i.e. the composition does not contain any (further) anionic or cationic surfactant(s).

Particularly preferred non-ionic surfactants in all embodiments of the present invention are selected from the group consisting of lauryl glucoside, glyceryl stearate, PEG-100 stearate, PEG-200 hydrogenated glyceryl palmate and PEG-7 glyceryl cocoate, most preferably from lauryl glucoside, PEG-200 hydrogenated glyceryl palmate and PEG-7 glyceryl cocoate.

Particularly preferred zwitterionic (amphoteric) surfactants in all embodiments of the present invention are selected from the group consisting of cocamidopropyl betaines.

The total amount of the at least one non-ionic surfactant in cleansing compositions according to the present invention, in particular when the composition does not comprise (i.e. is free of) one or more soaps, is preferably selected in the range from 7.5 to 35 wt.-%, preferably from 10 to 35 wt.-%, based on the total weight of the cleansing composition. Further suitable ranges are from 7.5 to 25 wt.-%, from 10 to 25 wt.-%, from 7.5 to 20 wt.-%, from 10 to 20 wt.-%, from 15-35 wt.-%, from 20-30 wt.-% and from 22-27 wt.-%.

The total amount of the at least one zwitterionic surfactant in cleansing compositions according to the present invention, in particular when the composition does not comprise (i.e. is free of) one or more soaps, is preferably selected in the range from 7.5 to 35 wt.-%, preferably from 10 to 35 wt.-%, based on the total weight of the cleansing composition. Further suitable ranges are from 7.5 to 25 wt.-%, from 10 to 25 wt.-%, from 7.5 to 20 wt.-%, from 10 to 20 wt.-%, from 15-35 wt.-%, from 20-30 wt.-% and from 22-27 wt.-%

It is furthermore advantageous, if the ratio (by weight) of the non-ionic to the zwitterionic surfactant(s) is selected in the range of 5:1 to 1:5, preferably 2.5:1 to 1:2.5.

Even more preferably, the non-ionic surfactant(s) is used in an excess, such as in an excess of at most 50 wt.-%, more preferably at most 40 wt.-%, based on the amount of the zwitterionic surfactant.

The (total) amount of surfactants in cleansing compositions according to the present invention is preferably selected in the range of from 10 to 75 wt.-%, from 20 to 75 wt.-%, from 25 to 75 wt.-%, more preferably in the range from 10 to 60 wt.-% or from to 60 wt.-%, from 30 to 60 wt.-%, most preferably in the range from 10 to 55 wt.-% or from 25 to 55 wt.-%, from 30 to 55 wt.-%, such as in the range of 10 to 40 wt.-% or from 30 to 40 wt.-%, based on the total weight of the cleansing composition. Further suitable ranges encompass from 10 to 30 wt.-% or from 10 to 25 wt.-%.

In a particularly preferred embodiment, the cleansing compositions according to the present invention comprise at least one, preferably two to three non-ionic surfactants, which are most preferably selected from lauryl glucoside; PEG-200 hydrogenated glyceryl palmate and PEG-7 glyceryl cocoate and one zwitterionic surfactant, preferably selected from the group consisting of alkyl amidopropyl betaines, most preferably the zwitterionic surfactant is cocoamidopropyl betaine.

In all embodiments of the present invention, the cleansing compositions according to the present invention preferably comprise at least 20 wt.-% of water, more preferably at least 35 wt.-% of water, even more preferably at least 40 wt.-% of water, most preferably at least 45 wt.-% of water, based on the total weight of said cleansing compositions. In particular, the water content of the cleansing compositions in all embodiments of the present invention is selected in the range from 20 to 75 w.-%, most preferably in the range from 20 to 60 wt-%, based on the total weight of the cleansing composition. Further suitable ranges encompass 25 to 60 wt. %, 25 to 55 wt.-%, 25 to 50 wt.-%, 30 to 60 wt. %, 30 to 55 wt.-%, 30 to 50 wt.-%, 40 to 60 wt. %, to 55 wt.-% and 40 to 50 wt.-%.

The cleansing compositions of the present invention may also include one or more optional ingredients such as a pearlescent or opacifying agent, a thickening agent, humectants, chelating agents, and additives which enhance their appearance, feel and fragrance, such as colorants, fragrances, preservatives, pH adjusting agents, and the like.

Commercially available pearlescent or opacifying agents which are capable of suspending water insoluble additives and/or which tend to indicate to consumers that the resultant product is a cleansing composition are suitable for use in this invention.

The pearlescent or opacifying agent may be present in an amount, based upon the total weight of the composition, of from about 1 to 10 wt.-%, preferably from about 1.5 to 7 wt.-%, and more preferably, from about 2 to 5 wt.-%.

Examples of suitable pearlescent or opacifying agents include, but are not limited to mono or diesters of (a) fatty acids having from about 16 to about 22 carbon atoms and (b) either ethylene or propylene glycol mono or diesters of (a) fatty acids having from about 16 to about 22 carbon atoms, (b) a polyalkylene glycol of the formula: HO-(JO)a-H, wherein J is an alkylene group having from about 2 to about 3 carbon atoms and a is 2 or 3; fatty alcohols containing from about 16 to about 22 carbon atoms; fatty esters of the formula: KCOOCH₂L, wherein K and L independently contain from about 15 to about 21 carbon atoms; inorganic solids insoluble in the cleansing composition, and mixtures thereof.

The pearlescent or opacifying agent may be introduced to the cleansing composition as a pre-formed, stabilized aqueous dispersion, such as that commercially available from Henkel Corporation of Hoboken, New Jersey under the tradename, “Euperlan PK-3000.” This material is a combination of glycol distearate (the diester of ethylene glycol and stearic acid), Laureth-4 (CH₃(CH₂)₁₀CH₂(OCH₂CH₂)₄OH) and cocamidopropyl betaine and preferably is in a weight percent ratio of from about 25 to about 30:about 3 to about 15: about 20 to about 25, respectively.

Commercially available thickening agents, which are capable of imparting the appropriate viscosity to the cleansing compositions are suitable for use in this invention. If used, the thickener should be present in the compositions in an amount sufficient to raise the Brookfield viscosity of the composition to a value of between about 500 to about 10,000 centipoise. Examples of suitable thickening agents nonexclusively include: mono or diesters of 1) polyethylene glycol of formula: HO—(CH₂CH₂O)₂H, wherein z is an integer from about 3 to about 200; and 2) fatty acids containing from about 16 to about 22 carbon atoms; fatty acid esters of ethoxylated polyols; ethoxylated derivatives of mono and diesters of fatty acids and glycerine; hydroxyalkyl cellulose; alkyl cellulose; hydroxyalkyl alkyl cellulose; and mixtures thereof. Preferred thickeners include polyethylene glycol ester, and more preferably PEG-150 distearate which is available from the Stepan Company of Northfield, Illinois or from Comiel, S.p.A. of Bologna, Italy under the tradename, “PEG 6000 DS”.

The total amount of thickener(s) in the cleansing composition is preferably selected in the range from 0 to 7 wt.-%, preferably from 1 to 5 wt.-%, most preferably from 2 to 4 wt.-%, based on the total weight of the cleansing composition. Further suitable ranges encompass 0 to 2.5 wt.-% and 0 to 1 wt.-% and 1 to 3 wt.-%.

The cleansing compositions according to the present invention advantageously further comprise one or more polyethylene glycols (PEGs) which can be used as binders, humectants and/or solvents. A particularly preferred polyethyleneglycol for the use in the cleansing compositions according to the present invention is PEG-32. When present, the amount of said PEG(s) in the cleansing composition according to the present invention is preferably selected in the range from 0.1 to 10 wt.-%, preferably from 1 to 7.5 wt.-%, most preferably from 2.5 to 7.5 wt.-%, based on the total weight of the cleansing composition. Further suitable ranges encompass 0 to 7.5 wt.-% and 1 to 7.5 wt.-% and 4 to 7.5 wt.-%.

Commercially available humectants, which are capable of providing moisturization and conditioning properties to the cleansing composition, are suitable for use in the cleansing compositions according to the present invention. Examples of suitable humectants nonexclusively include: 1) water soluble liquid polyols such as glycerol; 2) polyalkylene glycol of the formula: HO—(R″O)b—H, wherein R″ is an alkylene group having from about 2 to about 3 carbon atoms and b is an integer of from about 2 to about 10; 3) polyethylene glycol ether of methyl glucose of formula CH₃—C₆H₁₀O₅—(OCH₂CH₂)c-OH, wherein c is an integer from about 5 to about 25; 4) urea; 5) hyaluronic acid or derivatives thereof such as sodium hyaluronate and 6) mixtures thereof, with glycerol and hyaluronic acid or derivatives thereof being the preferred humectants.

Preferably, in all embodiments, the cleansing composition comprise at least one humectant. The amount of said at least one humectant, preferably glycerol and/or sodium hyaluronate, most preferably sodium hyaluronate is preferably selected in the range from 0 to 90 wt.-%, preferably from 2.5 to 40 wt.-%, most preferably from 4 to 10 wt.-%, based on the total weight of the cleansing composition. Further suitable ranges encompass 1 to 7.5 wt.-% and 2.5 to 7.5 wt.-%.

Examples of suitable chelating agents include those which are capable of protecting and preserving the compositions of this invention. Preferably, the chelating agent is ethylenediamine tetraacetic acid (“EDTA”), and more preferably is tetrasodium EDTA, available commercially from Dow Chemical Company of Midland, Michigan under the tradename, “Versene 100XL” or even disodium EDTA, commercially available from BASF under tradename “EDETA BD” and is present in an amount, based upon the total weight of the composition, from about 0 to 0.5 wt.-%, preferably from 0.01 to 0.25 wt.-%, more preferably from about 0.025 to 0.25 wt.-%, such as from 0.05 to 0.25 wt.-%.

In a still further advantageous aspect of the invention, the cleansing compositions of the present invention further comprise a preservative and/or a preservative booster such as e.g. quaternium-15. When present, the preservative respectively the preservative booster is preferably used in an amount of 0.01 to 2 wt. %, more preferably in an amount of 0.05 to 1.5 wt.-%, most preferably in an amount of 0.1 to 1.0 wt.-%, based on the total weight of the cleansing composition.

The cleansing compositions according to the present invention may further comprise a skin and/or hair conditioning and/or soothing actives such as e.g. allantoin, d- or dl-panthenol, ethyl panthenol, bisabolol, phytantriol, hyaluronic acid as well as any salts thereof such as sodium hyaluronate, vitamins and/or plant extracts. The amount of such skin actives, when present, is preferably selected in the range from 0.01 to 1 wt.-%, more preferably from 0.025 to 0.5 wt. %, most preferably from 0.05 to 0.25 wt.-%, based on the total weight of the cleansing composition.

The pH of the cleansing compositions of this invention is preferably maintained in the range from about 4.5 to about 10.5, and more preferably from about 5.0 to about 10.0.

The compositions of the present invention may furthermore be “substantially free” of oils or silicones. As used herein, “substantially free” shall mean that the cleansing composition contains, based upon the total weight of the composition, less than about 1 wt.-%, for example, less than about 0.5 wt.-% or less than about 0.2 wt.-% oils and/or silicones.

Particularly suitable cleansing compositions according to the present invention are rinse-off compositions, even more in particular liquid aqueous rinse-off compositions.

The term ‘rinse-off’ as used herein is defined as per the Regulation (EC) No. 1223/2009 of the European Parliament and of the Council of 30 Nov. 2009 on cosmetic products (recast), i.e. a cosmetic product or composition is a rinse-off one when it is intended to be removed after application on skin, hair or mucous membranes of a human subject. The rinse-off cosmetic composition presently disclosed may be a rinse-off, cosmetic cleansing composition, for example a rinse-off, cosmetic, personal care, cleansing composition.

Advantageously, the term ‘liquid’ as used herein means that the composition has a viscosity lower than about 40000 cPs, such as lower than about 30000 or lower than about 20000 cPs or lower than 15000 cPs, and greater than for example about 500 cPs, or greater than about 1000cPs or greater than about 2000 cPs, or greater than 5000 cPs or greater than 6500 cPs when measured at 20° C. and 1 atm by using a Brookfield viscometer at for example 20 or lower rpm. The choice of an appropriate spindle can be made by a skilled man based on e.g. information provided by viscometer manufacturers and the expected viscosity range of the composition to be tested.

Examples of the rinse-off cosmetic composition according to the present invention are shower gels (body shampoos), liquid soaps, wash gels, body washes, soap bars, foam baths, (hair) shampoos as well as shaving preparations. Said compositions are not edible or comestible, like toothpastes. Particularly preferred rinse-off compositions according to the present invention are liquid aqueous rinse off compositions such as shower gels (body shampoos), liquid soaps, wash gels, body washes foam baths and (hair) shampoos.

In a particular advantageous aspect, the cleansing compositions according to the present invention are free of any parabenes, benzethoniumchlorid, piroctone olamine, lauroylarginat, methylisothiazolinon, chlormethylisothiazolinon, bronopol, benzalkoniumchloride, formaldehyd releasing compounds, salicylic acid, triclosan, DMDM hydantoin, chlorphenesin and IPBC (lodopropinylbutyl carbamate), such as in particular free of methylchloroisothiazolinone.

In a further particular embodiment, the invention also relates to a method for washing the skin and/or hair, said method comprising applying a cleansing composition according to the present invention with all the definitions and preferences as given herein to the skin and/or hair, followed by washing (i.e. rubbing the composition onto the skin to produce foaming) and then rinsing (i.e. rinsing the composition off the skin).

In another embodiment, the present invention also relates to the use of a cleansing composition according to the present invention with all the definitions and preferences as given herein for washing the skin and/or the hair.

The cleansing composition of the present invention may be used on the body in conjunction with any personal cleansing implement known in the art such as a washcloth, a mesh or apertured film, pouf, sponge, brush and the like. The composition may be marketed together with one or more of such implements in a kit.

The following examples are provided to further illustrate the compositions and effects of the present invention. These examples are illustrative only and are not intended to limit the scope of the invention in any way.

### EXAMPLES

### 1.Foaming Properties of HMO's

### Procedure:

Add 2.5 g of a 1% solution of a sensitive skin cleanser in a tube (Diameter 2.2 cm, height 15 cm)
Add 2.5 g of water (reference) or a 1% aqueous solution of the respective HMO
Note the height of liquid in the tube
Shake 15s (close the test tube with a stopper)
Note the max height of the mixture (based on the foam produced)
Result is the difference between the maximum height of the reference versus the respective test sample after shaking.

### HMO's Abbreviation

2′-FL: 2′-Fucosyllactose
3-FL: 3-Fucosyllactose
3′SL: 3′-Sialyllactose sodium salt
6′SL: 6′-Sialyllactose sodium salt
LNnT: Lacto-N-neotetraose
LNT: Lacto-N-tetraose
FL/DFL: 2′-Fucosyllactose/Difucosyllactose mixture (75% 2′-FL; 5% DFL)

TABLE 1a
Formulation sensitive skin cleanser

| Ingredients | wt-% |
|---|---|
| Water Dem | ad 100 |
| Allantoin | 0.1 |
| Chelating agent (Edeta ® BD) | 0.05 |
| Hyaluronic acid | 0.01 |
| Diols | 6 |
| Preservative(s) | 0.1 |
| Zwitterionic surfactant (cocamidopropyl betaine) | 25 |
| Non-ionic surfactant (lauryl glucoside; PEG-200 hydrogenated | 25 |
| glyceryl palmate; PEG-7 glyceryl cocoate) | |
| Citric Acid | 0.3 |

TABLE 1b
Results: Foam volume

| # | HMO | Foam volume [mm] | %* |
|---|---|---|---|
| Reference | — | 19 | 100 |
| Inv-1 | 2′-FL | 20 | 105 |
| Inv-2 | 3′-SL | 26 | 137 |
| Inv-3 | 3-FL | 29 | 153 |
| Inv-4 | 6′-SL | 24 | 126 |
| Inv-5 | LNnT | 26 | 137 |
| Inv-6 | LNT | 25 | 132 |
| Inv-7 | 2′-FL/DFL | 25 | 132 |

*based on reference set at 100%

The samples according to present invention showed a significantly higher foam volume highlighting the excellent foam boosting properties of the respective HMO.

The same experiment has been repeated using 0.5% and 2% HMO solutions, respectively 0.5% and 2% maltose solutions (reference). The results are depicted in table 1.1.c and table 1.1.d below.

TABLE 1.1.c
Results: Foam volume using 2% HMO solution
respectively 2% maltose solutions

| # | HMO | 2%* |
|---|---|---|
| Reference (control) | — | 100% |
| Reference maltose | — | 117% |
| Inv-8 | 3′SL | 121% |
| Inv-9 | LNnT | 121% |
| Inv-10 | LNT | 133% |

*based on reference set at 100%

TABLE 1.1.d
Results: Foam volume using 0.5% HMO solution
respectively 0.5% maltose solutions

| # | HMO | 0.5%* |
|---|---|---|
| Reference (control) | — | 100% |
| Reference maltose | — | 108% |
| Inv-11 | 2′-FL/DFL | 112% |
| Inv-12 | LnNT | 112% |

*based on reference set at 100%

Compared to the reference, also these samples showed an improved foaming highlighting the foam boosting properties of the respective HMO over a wide concentration range. The use of maltose did not result in equally advantageous results.

1. A sulfate-free cleansing composition comprising at least one zwitterionic surfactant containing at least 8 carbon atoms and one or more human milk oligosaccharide.

2. The cleansing composition according to claim 1, wherein the amount of the at least one human milk oligosaccharide is selected in the range from 0.01 to 10 wt.-%, preferably in the range from 0.1 to 7.5 wt.-%, most preferably in the range from 0.2 to 5 wt.-%, based on the total weight of the composition.

3. The cleansing composition according to claim 1, wherein the at least one human milk oligosaccharide is selected from the group consisting of 2′-fucosyllactose, 3-fucosyllactose, difucosyllactose, lacto-N-fucopentaose I, 3′sialyllactose sodium salt, 6′sialyllactose sodium salt, lacto-N-neotetraose and tacto-N-tetraose as well as mixtures thereof, preferably from 3-fucosyllactose, difucosyllactose, 3′sialyllactose sodium salt, 6′sialyllactose sodium salt, lacto-N-neotetraose and lacto-N-tetraose as well as mixtures thereof, most preferably from 3-fucosyllactose, 3′sialyllactose sodium salt, and lacto-N-neotetraose.

4. The cleansing composition according to claim 1, wherein the composition further comprises at least one non-ionic surfactant.

5. The cleansing composition according to claim 4, wherein the at least one non-ionic surfactant is selected from the group consisting of alkyl (poly)glucosides, (poly)glyceryl fatty acid esters and polyethyleneglycol (PEG) based surfactants.

6. The cleansing composition according to claim 4, wherein the at least one non-ionic surfactant is selected from the group consisting of lauryl glucoside, glyceryl stearate, PEG-100 stearate, PEG-200 hydrogenated glyceryl palmate and PEG-7 glyceryl cocoate.

7. The cleansing composition according to claim 1, wherein the at least one one zwitterionic surfactant is selected from the group consisting of alkyl amidopropyl betaines, preferably the at least one zwitterionic surfactant is cocamidopropyl betaine.

8. The cleansing composition according to claim 1, wherein the amount of the at least one surfactant is selected in the range from to 75 wt.-%, preferably in the range from 30 to 60 wt.-%, most preferably in the range from to 55 wt.-%, based on the total weight of the cleansing composition.

9. The cleansing composition according to claim 1, wherein the cleansing composition comprises at least 35 wt.-% of water, preferably at least 40 wt.-% of water, most preferably at least 45 wt.-% of water, based on the total weight of the cleansing composition.

10. The cleansing composition according to claim 1, wherein the cleansing composition is a rinse-off composition.

11. The cleansing composition according to claim 10, wherein the cleansing composition is a shower gel, a liquid soap, a wash gel, a body wash, a soap bar, a foam bath, a shampoo or a shaving preparations.

12. The cleansing composition according to claim 1, wherein the cleansing composition further comprises at least one humectant, preferably sodium hyaluronate.

13. A method of increasing the foaming properties of sulfate-free cleansing compositions comprising at least one surfactant, said method comprising the step of adding at least one human milk oligosaccharide into said cleansing composition.

14. Use of use of at least one human milk oligosaccharide as foam enhancing agent and/or foam booster.

15. Use according to claim 14, to enhance the foam volume.
